(19) United States
(12) Patent Application Publication
(10) Pub. No.: US 2024/0342133 A1
(43) Pub. Date: Oct. 17, 2024

(54) TOPICAL STATIN FORMULATION FOR TREATMENT OF LICHEN SCLEROSUS AND PSORIASIS

(71) Applicant: TBT PHARMA, INC, Fort Collins, CO (US)

(72) Inventor: IAN TOLFREE, NEW YORK, NY (US)

(21) Appl. No.: 18/132,985

(22) Filed: Apr. 11, 2023

(51) Int. Cl. A61K 31/366; A61K 9/00; A61K 9/127

(52) U.S. Cl. CPC A61K 31/366; A61K 9/0014; A61K 9/1272

(57) ABSTRACT

The invention relates to methods and compositions for treating lichen sclerosus and/or psoriasis by administering to a patient in need thereof a topical pharmaceutical formulation comprising simvastatin 1.07-5.0% w/w/in a non-aqueous, low pH, biphasic liposomal-PEG carrier.

### FIELD OF THE INVENTION

The embodiments described herein relate generally to a method for treating lichen sclerosus and/or psoriasis by administering to a patient in need thereof a topical pharmaceutical formulation comprising simvastatin 1.0%-5.0% w/w/in a non-aqueous, low pH, biphasic liposomal-PEG carrier.

### BACKGROUND

Lichen sclerosus is a skin disease characterized as a chronic inflammatory disorder which causes itchy, sometimes painful, white patches on genitals or other parts of the body. Lichen sclerosus is believed to be non-infectious but also increases the risk of squamous cell skin cancer. Despite extensive research the etiology of the disease is still unknown

Psoriasis is a skin disease characterized as a chronic relapsing inflammatory disease which affects up to 3% of the population. Symptoms include the development of scaling erythematous plaques of various sizes and forms which may extend to large areas of the skin. Psoriasis is composed of two main processes: cellular hyperproliferation and inflammation. Despite extensive research the etiology of the disease is still unknown.

Psoriasis has been treated by a number of methods including topical applications consisting of tar derivatives, steroids, vitamin D and its derivatives or vitamin A and its derivatives (J. P. Callen, Drug Therapy, April 1987, pp. 29-35). These therapies are not entirely successful and are often accompanied by undesirable side effects. Side effects from steroids are particularly deleterious. Other published therapies include phototherapy, and also the systemic administration of steroids, methotrexate and cyclosporine. All of these therapies are associated with deleterious side effects.

Atopic dermatitis is a chronic skin condition affecting patients of all ages. There is about 4% incidence of atopic dermatitis from birth to 7 years (Halpern et al., J. Allergy Clin. Immunol. 51:139-151 (1973). In childhood, it is characterized by papules, erythema, thickening and lichenification. In the adolescent, the main symptoms are thickening and lichenification with erythema and scaling. Pruritis is a general feature of the disease. Systemic therapy includes antihistamine drugs and steroids, but the latter are reserved for unmanageable cases and used for the shortest period possible. Topical therapy includes fluorinated and fluorochlorinated corticosteroid preparations, but striae and cataracts are likely complications. Clearly there is as yet no satisfactory and safe drug treatment for atopic dermatitis.

Xanthine derivatives have been proposed for the treatment of psoriasis and atopic dermatitis. U.S. Pat. No. 4,141,976 proposes certain pharmaceutical preparations for the topical treatment of psoriasis. Among the compounds described are certain substituted alkylxanthine derivatives and substituted thioxanthines. However data demonstrating effectiveness was shown only for RO 20-1724 d, 1-4-(3-butoxy-4-methoxybenzyl)-2-imidazolidinone, but which is not a xanthine derivative. No data was provided showing xanthines were topically effective when administered to patients suffering from proliferative skin disease.

Accordingly, a need exists for an alternative topical formulation for treatment of lichen sclerosus, psoriasis and atopic dermatitis.

### SUMMARY

The embodiments described herein are directed to a method for treating lichen sclerosus and/or psoriasis by administering to a patient in need thereof a topical pharmaceutical formulation comprising simvastatin 1.0%-5.0% w/w/in a non-aqueous, low pH, biphasic liposomal-PEG carrier.

In another embodiment, a method for treating lichen sclerosus and/or psoriasis by administering to a patient in need thereof a topical pharmaceutical formulation comprising a statin 1.0%-5.0% w/w/in a non-aqueous, low pH, biphasic liposomal-PEG carrier.

The embodiments described herein are directed to a topical pharmaceutical formulation comprising simvastatin 1.0%-5.0%, and preferably 2.5%-3.5% w/w/in a non-aqueous, low pH, biphasic liposomal-PEG carrier.

In another embodiment, the invention is a topical pharmaceutical formulation comprising a statin 1.0%-5.0%, and preferably 3.5% w/w/in a non-aqueous, low pH, biphasic liposomal-PEG carrier.

In another embodiment, the statin is selected from the group consisting of simvastatin, cerivastatin, lovastatin, pravastatin, atorvastatin, fluvastatin, NK-104, and mixtures thereof.

Disclosed are embodiments to a topical pharmaceutical composition comprising a statin for use in the topical treatment of psoriasis and atopic dermatitis.

The pharmaceutically acceptable carrier of the compositions according to the invention may contain any of the components which are used in topical compositions and are well known to those skilled in the art.

Any of the embodiments of the invention may employ a penetration enhancer such as urea, lactic acid, ammonium lactate, salicylic acid or a C3-C12-straight chain alkanoic acid.

Any of the embodiments of the invention may be in the form of lotions, creams, ointments and gels, and also in the form of sprayable aerosols. Preferred formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, typically polyoxy-40-stearate.

The preferred active compound according to the invention is simvastatin.

Any of the embodiments may include compositions having a pH from 3.8-4.5.

Any of the embodiments may include compositions comprising a water-in-oil emulsion.

Any of the embodiments may include compositions comprising a mixture of ceramides (˜40-50% by weight), hyaluronic acid (˜10-25% by weight), cholesterol (˜10-25% by weight), and free fatty acids (˜5-15% by weight).

Any of the embodiments may include compositions comprising an equimolar mixture of ceramides (˜50% by weight), cholesterol (˜25% by weight), and free fatty acids (˜15% by weight).

Any of the embodiments may include a composition comprising a mixture of buffered glycolic acid and ammonia.

Any of the embodiments may include a composition comprising a mixture of buffered glycolic acid and ammonia, in a formulation comprising aqua, isohexadecane, cetearyl isononanoate, dicaprylyl ether, sorbitan oleate, glycerin, hydrogenated vegetable oil, polyglyceryl-3 polyricinoleate, sucrose polystearate, magnesium sulfate, parfum, tocopherol, limonene, Helianthus annuus seed oil, linalool, BHT, citral.

Any of the embodiments may include compositions comprising a mixture of ceramides (˜40-50% by weight), hyaluronic acid (˜10-25% by weight), cholesterol (˜10-25% by weight), and free fatty acids (˜5-15% by weight), with buffered glycolic acid and ammonia, in a formulation comprising aqua, isohexadecane, cetearyl isononanoate, polyricinoleate, sucrose polystearate, magnesium sulfate, parfum, tocopherol, limonene, Helianthus annuus seed oil, linalool, BHT, citral.

### BRIEF DESCRIPTION OF THE FIGURES

FIG. 1. Relative cortisol levels in healthy skin vs. chronic wounds.

FIG. 2. The feedback loop that keeps a chronic wound from becoming acute.

FIG. 3. C-myc expression in ex-vivo cultures before and after treatment with mevastatin.

FIG. 4. Cortisol levels in organotype wound models after treatment with mevastatin.

FIG. 5. Epithelialization rates are various mevastatin doses in a porcine full thickness wound.

FIG. 6. Histology sections of optimal dose at day 10 show increased epithelialization and vascularization.

FIG. 7. Percent vascularity at day 10 determined by PD31 stain.

FIG. 8. Ex-vivo wound cultures treated with meva and simvastatin show a drastic reduction in MRSA load after 24 hours.

### DETAILED DESCRIPTION OF THE INVENTION

Disclosed are embodiments to a method for treating lichen sclerosus and/or psoriasis by administering to a patient in need thereof a topical pharmaceutical formulation comprising simvastatin 1.0%-5.0% w/w/in a non-aqueous, low pH, biphasic liposomal-PEG carrier.

In another embodiment, a method for treating lichen sclerosus and/or psoriasis by administering to a patient in need thereof a topical pharmaceutical formulation comprising a statin 1.0%-5.0% w/w/in a non-aqueous, low pH, biphasic liposomal-PEG carrier.

The embodiments described herein are directed to a topical pharmaceutical formulation comprising simvastatin 1.0%-5.0%, and preferably 2.5%-3.5% w/w/in a non-aqueous, low pH, biphasic liposomal-PEG carrier.

In another embodiment, the invention is a topical pharmaceutical formulation comprising a statin 1.0%-5.0%, and preferably 3.5% w/w/in a non-aqueous, low pH, biphasic liposomal-PEG carrier.

Preferably the concentration of the statin compound is about 0.5%-5% (w/w). In some embodiments, the concentration of statin is preferably 2% (w/w).

In another embodiment, the statin is selected from the group consisting of simvastatin, cerivastatin, lovastatin, pravastatin, atorvastatin, fluvastatin, NK-104, and mixtures thereof.

The pharmaceutically acceptable carrier of the compositions according to the invention may contain any of the components which are used in topical compositions and are well known to those skilled in the art.

Any of the embodiments of the invention may employ a penetration enhancer such as urea, lactic acid, ammonium lactate, salicylic acid or a C3-C12-straight chain alkanoic acid.

Any of the embodiments of the invention may be in the form of lotions, creams, ointments and gels, and also in the form of sprayable aerosols. Preferred formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, typically polyoxy-40-stearate.

The preferred active compound according to the invention is simvastatin.

Any of the embodiments may include compositions having a pH from 3.8-4.5.

Any of the embodiments may include compositions comprising a water-in-oil emulsion.

Any of the embodiments may include compositions comprising a mixture of ceramides (˜40-50% by weight), hyaluronic acid (˜10-25% by weight), cholesterol (˜10-25% by weight), and free fatty acids (˜5-15% by weight).

Any of the embodiments may include compositions comprising an equimolar mixture of ceramides (˜50% by weight), cholesterol (˜25% by weight), and free fatty acids (˜15% by weight).

Any of the embodiments may include a composition comprising a mixture of buffered glycolic acid and ammonia.

Any of the embodiments may include a composition comprising a mixture of buffered glycolic acid and ammonia, in a formulation comprising aqua, isohexadecane, cetearyl isononanoate, dicaprylyl ether, sorbitan oleate, glycerin, hydrogenated vegetable oil, polyglyceryl-3 polyricinoleate, sucrose polystearate, magnesium sulfate, parfum, tocopherol, limonene, Helianthus annuus seed oil, linalool, BHT, citral.

Any of the embodiments may include compositions comprising a mixture of ceramides (˜40-50% by weight), hyaluronic acid (˜10-25% by weight), cholesterol (˜10-25% by weight), and free fatty acids (˜5-15% by weight), with buffered glycolic acid and ammonia, in a formulation comprising aqua, isohexadecane, cetearyl isononanoate, dicaprylyl ether, sorbitan oleate, glycerin, hydrogenated vegetable oil. polyglyceryl-3 polyricinoleate, sucrose polystearate, magnesium sulfate, parfum, tocopherol, limonene, Helianthus annuus seed oil, linalool, BHT, citral.

### Definitions

The terminology used herein is for the purpose of describing particular embodiments only and is not intended to limit the full scope of the claims. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a”, “an” and “the” are intended to include the plural forms as well, unless the context clearly indicates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” etc.). Similarly, the terms “comprises” and/or “comprising,” when used in this specification, specify the presence of stated features, integers (or fractions thereof), steps, operations, elements, and/or components, but do not preclude the presence or addition of one or more other features, integers (or fractions thereof), steps, operations, elements, components, and/or groups thereof. As used in this document, the term “comprising” means “including, but not limited to.”

As used herein the term “and/or” includes any and all combinations of one or more of the associated listed items. It should be understood that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All ranges disclosed herein also encompass any and all possible subranges and combinations of subranges thereof unless expressly stated otherwise. Any listed range should be recognized as sufficiently describing and enabling the same range being broken down into at least equal subparts unless expressly stated otherwise. As will be understood by one skilled in the art, a range includes each individual member.

The term “liposome” as used herein means a vesicle including one or more concentrically ordered lipid bilayer(s) encapsulating a liquid phase, when in an liquid environment. Formation of such vesicles requires the presence of “vesicle-forming lipids” which are defined herein as amphipathic lipids capable of either forming or being incorporated into a bilayer structure. The term includes lipids that are capable of forming a bilayer by themselves or when in combination with another lipid or lipids. An amphipathic lipid is incorporated into a lipid bilayer by having its hydrophobic moiety in contact with the interior, hydrophobic region of the bilayer membrane and its polar head moiety oriented towards an outer, polar surface of the membrane. Hydrophilicity arises from the presence of functional groups such as hydroxyl, phosphate, carboxyl, sulfate, amino or sulfhydryl groups. Hydrophobicity results from the presence of a long chain of aliphatic hydrocarbon groups.

Liposomes can be categorized into multilamellar vesicles, multivesicular liposomes, unilamellar vesicles and giant liposomes. Multilamellar liposomes (also known as multilamellar vesicles or “MLV”) contain multiple concentric bilayers within each liposome particle, resembling the “layers of an onion”.

Multivesicular liposomes consist of lipid membranes enclosing multiple non-concentric aqueous chambers.

Unilamellar liposomes enclose a single internal aqueous compartment.

Single bilayer (or substantially single bilayer) liposomes include small unilamellar vesicles (SUV) and large unilamellar vesicles (LUV). LUVs and SUVs range in size from about 50 to 500 nm and 20 to 50 nm respectively.

Any suitable vesicle-forming lipid may be utilized in the practice of this invention as judged by one of skill in the art. This includes phospholipids such as phosphatidylcholine (PC), phosphatidylglycerol (PG), phosphatidylinositol (PI), phosphatidic acid (PA), phosphatidyethanolamine (PE) and phosphatidylserine (PS); sterols such as cholesterol; glycolipids; sphingolipids such as sphingosine, ceramides, sphingomyelin, and glycosphingolipids (such as cerebrosides and gangliosides).

Suitable phospholipids may include one or two acyl chains having any number of carbon atoms, between about 6 to about 24 carbon atoms, selected independently of one another and with varying degrees of unsaturation. Thus, combinations of phospholipid of different species and different chain lengths in varying ratios may be selected. Mixtures of lipids in suitable ratios, as judged by one of skill in the art, may also be used.

Liposomes for use in the present invention may be generated using a variety of conventional techniques. These techniques include: the ether injection method; the surfactant method; the Ca2+ fusion method; the freeze-thaw method; the reverse-phase evaporation method; the ultrasonic treatment method; the ethanol injection method; the extrusion method; the French press method; or any other technique described herein or known in the art.

Different techniques may be appropriate depending on the type of liposome desired. For example, small unilamellar vesicles (SUVs) can be prepared by the ultrasonic treatment method, the ethanol injection method, or the French press method, while multilamellar vesicles (MLVs) can be prepared by the reverse-phase evaporation method or by the simple addition of water to a lipid film followed by dispersal by mechanical agitation. LUVs may be prepared by the ether injection method, the surfactant method, the Ca2+ fusion method, the freeze-thaw method, the reverse-phase evaporation method, the French press method or the extrusion method.

In some embodiments, LUVs are prepared according to the extrusion method. The extrusion method involves first combining lipids in chloroform to give a desired molar ratio. A lipid marker may optionally be added to the lipid preparation. The resulting mixture is dried under a stream of nitrogen gas and placed in a vacuum pump until the solvent is substantially removed. The samples are then hydrated in an appropriate buffer or mixture of therapeutic agent or agents. The mixture is then passed through an extrusion apparatus (e.g. Extruder, Northern Lipids, Vancouver, BC) to obtain liposomes of a defined size. Average liposome size can be determined by quasi-elastic light scattering or photon correlation spectroscopy or dynamic light scattering or various electron microscopy techniques (such as negative staining transmission electron microscopy, freeze fracture electron microscopy or cryo-transmission electron microscopy).

The resulting liposomes may be run down a Sephadex™ CLAB column or similar size exclusion chromatography column equilibrated with an appropriate buffer in order to remove unencapsulated drug or to create an ion gradient by exchange of the exterior buffer. Subsequent to generation of an ion gradient, LUVs may encapsulate therapeutic agents as set forth herein.

Any of the embodiments herein may employ a liposome are prepared to be “cholesterol free”, meaning that such lipid-based vehicles contain “substantially no cholesterol,” or contain “essentially no cholesterol.” The term “cholesterol-free” as used herein with reference to a liposome means that the liposome is prepared in the absence of cholesterol, or contains substantially no cholesterol, or that the vehicle contains essentially no cholesterol. The term “substantially no cholesterol” allows for the presence of an amount of cholesterol that is insufficient to significantly alter the phase transition characteristics of the liposome (typically less than 20 mol % cholesterol). 20 mol % or more of cholesterol broadens the range of temperatures at which phase transition occurs, with phase transition disappearing at higher cholesterol levels. A liposome having substantially no cholesterol may have about 15 or less, or about 10 or less mol % cholesterol. The term “essentially no cholesterol” means about 5 or less mol %, or about 2 or less mol %, or about 1 or less mol % cholesterol. In some embodiments, no cholesterol will be present or added when preparing “cholesterol-free” liposomes. The presence or absence of cholesterol may influence the ability of the micelle-solubilized compound that can be stably incorporated into the liposome bilayer and may influence retention of that compound after incorporation.

Liposomes may range from any value between about 50 nm to about 1 um in diameter. In some embodiments, liposomes in a liposomal composition according to the invention may be less than about 200 nm in diameter, or less than about 160 nm in diameter, or less than about 140 nm in diameter.

In some embodiments, liposomes in a liposomal composition according to the invention may be substantially uniform in size, for example, 10% to 100%, or more generally at least 10%, 20%, 30%, 40%, 50, 55% or 60%, or at least 65%, 75%, 80%, 85%, 90%, or 95%, or as much as 96%, 97%, 98%, 99%, or 100% of the liposomes in the liposomal composition may be between the size values indicated herein. Liposomes may be sized by extrusion through a filter (e.g. a polycarbonate filter) having pores or passages of the desired diameter.

In some embodiments, liposomes may include a hydrophilic moiety. Grafting a hydrophilic moiety to the surface of liposomes can “sterically stabilize” liposomes. Typically, the hydrophilic moiety is conjugated to a lipid component of the liposome, forming a hydrophilic polymer-lipid conjugate. The term “hydrophilic polymer-lipid conjugate” refers to a lipid, e.g., a vesicle-forming lipid, covalently joined at its polar head moiety to a hydrophilic polymer, and is typically made from a lipid that has a reactive functional group at the polar head moiety in order to attach the polymer. Suitable reactive functional groups are for example, amino, hydroxyl, carboxyl or formyl groups. The lipid may be any lipid described in the art for use in such conjugates. The lipid may be a phospholipid having one or two acyl chains including between about 6 to about 24 carbon atoms in length with varying degrees of unsaturation.

In some embodiments, the lipid in the conjugate may be a PE, such as of the distearoyl form. The polymer may be a biocompatible polymer characterized by a solubility in water that permits polymer chains to effectively extend away from a liposome surface with sufficient flexibility that produces uniform surface coverage of a liposome. Such a polymer may be a polyalkylether, including polyethylene glycol (PEG), polymethylene glycol, polyhydroxy propylene glycol, polypropylene glycol, polylactic acid, polyglycolic acid, polyacrylic acid and copolymers thereof.

The polymer may have an average molecular weight of any value between about 350 and about 10,000 daltons. In alternative embodiments, the phospholipids may be selected from poly (ethylene glycol) (PEG) modified phospholipids. The average molecular weight of the PEG may be any value between about 500 to about 10,000 Daltons.

Combinations of PEG phospholipid of different species and different chain lengths in varying ratios may be selected. Combinations of phospholipids and PEG phospholipids may also be selected. The conjugate may be prepared to include a releasable lipid-polymer linkage such as a peptide, ester, or disulfide linkage.

Any of the embodiments of the invention may employ liposomes that include a statin, prepared by conventional “active” or “passive” loading methods. For example, a statin can be mixed with vesicle-forming lipids and be incorporated within a lipid film, such that when the liposome is generated, the statin is incorporated or encapsulated into the liposome. Thus, if the statin is substantially hydrophobic, it will be encapsulated in the bilayer of the liposome. Alternatively, if the statin is substantially hydrophilic, it will be encapsulated in the aqueous interior of the liposome. The statin may be soluble in aqueous buffer or aided with the use of detergents or ethanol. The liposomes can subsequently be purified, for example, through column chromatography or dialysis to remove any unincorporated therapeutic agent.

Any of the embodiments of the invention may employ liposomes prepared and formed in advance i.e., be “pre-formed” liposomes. Pre-formed liposomes may be used to prepare the liposomal formulations according to the invention. Such pre-formed liposomes may include an agent, such as a therapeutic agent, or an agent may be added to pre-formed liposomes prior to preparation of liposomal compositions according to the invention e.g., prior to combination with a micelle containing an agent. In some embodiments, pre-formed liposomes do not include a hydrophilic moiety. Pre-formed liposomes are available from various commercial contract pharmaceutical companies with expertise in the art of preparing liposomes.

The term “micelle” as used herein means a self-assembled lipid arrangement without an aqueous phase and are generally <50 nm in mean diameter. Micelles may be spherical or tubular and form spontaneously at or above the critical micelle concentration (CMC). In general, micelles are in equilibrium with the monomers under a given set of physical conditions such as temperature, ionic environment, concentration, etc. Formation of a micelle requires the presence of “micelle-forming compounds,” which include amphipathic lipids (e.g., a vesicle-forming lipid as described herein or known in the art), lipoproteins, detergents, non-lipid polymers, or any other compound capable of either forming or being incorporated into a micellar structure. Thus, a micelle-forming compound includes compounds that are capable of forming a micelle by themselves or when in combination with another compound, and may be polymer micelles, block co-polymer micelles, polymer-lipid mixed micelles, or lipid micelles. A micelle-forming compound, in an aqueous environment, generally has a hydrophobic moiety in the interior of the micelle, and a polar head moiety oriented outwards into the aqueous environment. Hydrophilicity generally arises from the presence of functional groups such as hydroxyl, phosphate, carboxyl, sulfate, amino or sulfhydryl groups. Hydrophobicity generally results from the presence of a long chain of aliphatic hydrocarbon groups. A micelle may be prepared from lipoproteins or artificial lipoproteins including low density lipoproteins, chylomicrons and high density lipoproteins.

The term “micelles” refers to units having a range from about 5 nm to about 50 nm in diameter. In some embodiments, micelles may be less than about 50 nm in diameter, or less than about 30 nm in diameter, or less than about nm in diameter.

The term “hydrophilic polymer-lipid conjugate” refers to a lipid, e.g., a vesicle-forming lipid, covalently joined at its polar head moiety to a hydrophilic polymer, and is typically made from a lipid that has a reactive functional group at the 20 polar head moiety in order to attach the polymer. Examples include polyethylene glycol (PEG), polyvinyl alcohol, polylactic acid, polyglycolic acid, polyvinylpyrrolidone, polyacrylamide, polyglycerol, or synthetic lipids with polymeric head groups. For example, a distearoyl-phosphatidylethanolamine covalently bonded to a PEG alone, or in further combination with phosphatidylcholine (PC), may be used to produce a micelle according to the invention.

Any of the embodiments of the invention may employ a molecular weight of the PEG having a value between about 500 Daltons to about 10,000 Daltons, inclusive, for example, 1000, 2000, 4000, 6000, 8000, etc. The hydrophilic polymer-lipid conjugate will be dependent on the molecular weight of the PEG as well as the lipid anchor and the added components used when preparing mixed micelles (e.g. PEG modified distearoyl-phosphatidylethanolamine and PC).

The term “ceramide” refers to a family of waxy lipid molecules. A ceramide is composed of N-acetylsphingosine and a fatty acid. Ceramides are found in high concentrations within the cell membrane of eukaryotic cells, since they are component lipids that make up sphingomyelin, one of the major lipids in the lipid bilayer. As a general rule of thumb, the epidermal lipid matrix is composed of an equimolar mixture of ceramides (˜50% by weight), cholesterol (˜25% by weight), and free fatty acids (˜15% by weight), with smaller quantities of other lipids also being present. Specific ceramides contemplated herein include dihydroceramide ADS, dihydroceramide NDS, 6-hydroxyceramide AH, 6-hydroxyceramide EOH, 6-hydroxyceramide NH, phytoceramide, phytoceramide NP, ceramide AS, ceramide EOS, and ceramide NS. Ceramide nomenclature includes: “E” refers to ester linked fatty acid; “O” refers to amide-linked fatty acid (omega hydroxy); “S” refers to a sphingosine base; “P” refers to the phytosphingosine base; “NP” refers to N-stearoyl phytosphingosine; “AP” refers to α-hydroxy-N-stearoylphytosphingosine

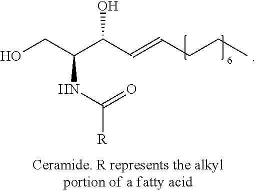

The term “hyaluronic acid” refers to an anionic, nonsulfated glycosaminoglycan that occurs as polymers of disaccharide monomers. Hyaluronic acid is major component of the extracellular matrix, hyaluronic acid has a key role in tissue regeneration, inflammation response, and angiogenesis, which are phases of wound repair. Hyaluronic acid combines with water and swells to form a gel, making it useful in skin treatments; its effect lasts for about 6 to 12 months, and treatment has regulatory approval from the US Food and Drug Administration.

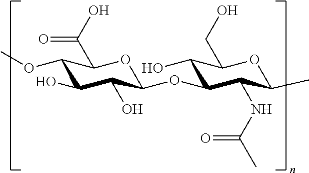

The term “cholesterol” refers to a lipid “sterol” molecule found in the stratum corneum outermost layer of epidermis, and functions to form, with ceramides and free fatty acids, a water-impermeable barrier for the skin.

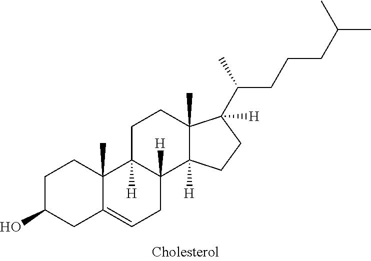

The term “free fatty acids” or “FFA” refers to non-esterified fatty acids, and in the skin, refers to C12-C26 saturated and unsaturated fatty acids, with C24 and C26 being most common. FFA contemplated herein include: Dodecanoic acid (C12:0); Myristic acid (C14:0); Palmitic acid (C16:0); Stearic acid (C18:0); Eicosanoic (arachidic) acid (C20:0); Behenic acid (C22:0); Lignoceric acid (C24:0); Hexascosanoic acid (C26:0); Monounsaturated fatty acids (MUFA) including Myristoleic acid (C14:1, n−5); Palmitoleic acid (C16:1, n−7); Sapienic acid (C16:1, n−10); Oleic acid (C18:1, n−9); Polyunsaturated fatty acids (PUFA) including Linoleic acid (C18:2, n−6); Arachidonic acid (C20:4, n−6); Eicosapentaenoic acid (C20:5, n−3); and Docosahexaenoic acid (C22:6, n−3).

The term pH refers to the acidity of the skin and the acidity of the compositions described herein. pH refers to the logarithmic level of hydrogen ion concentration in a mixture or solution. Human skin is known to use a low pH, e.g. pH 3.8-pH 4.5, to protect against bacteria, fungi, viruses, and other microorganisms. Using a high pH skin treatment (cream, lotion, foam, etc.) can add unwanted stress to an already damaged, infected, or inflamed area, requiring the skin to expend precious resources to re-establish its preferred lower pH in addition to managing the healing processes. Using a pH-appropriate skin treatment delivery vehicle for simvastin increases effectiveness over prior formulations.

The term “statin” or “statins” refer in a preferred embodiment to Simvastatin (brand names ZOCOR, LIPEX), and Simvastatin+Ezetimibe combination therapy (brand name VYTORIN), but also include, and are not limited to, Atorvastatin (brand names LIPITOR, TORVAST), Cerivastatin (brand names LIPOBAY, BAYCOL), Fluvastatin (brand names Lescol, Lecol XL), Lovastatin (brand names MEVACOR, ALTOCOR, ALTOPREY), Mevastatin (naturally occurring in organisms including, but not limited to, oyster mushrooms and Monascus purpureus), Pitavastatin (brand names LOVALO, PITAVA), Pravastatin (brand names PRAVACHOL, SELEKTINE, LIPOSTAT), Rosuvastatin (brand name CRESTOR), Lovastatin+Niacin combination therapy (brand name ADVICOR), Atorystatin+Amlidipine combination therapy (brand name CADUET), and Simvastatin+Niacin combination therapy (brand name SIMCOR).

Statins can be sub-grouped according to their hydrophobicity or hydrophilicity. Simvastatin (MW 418 Da), atorvastatin (MW 604 Da), cerivastatin (MW 481 Da), fluvastatin (MW 433 Da), and lovastatin (MW 404 Da) are hydrophobic. Pravastatin (Molecular weight (MW) 446 Da) and rosuvastatin (MW 500 Da) are hydrophilic. Hydrophobic statins easily diffuse through the cell membrane.

### Statin Compounds Useful in the Invention

Any of the embodiments of the invention may employ statin compounds useful in the compositions of the invention having the formula:

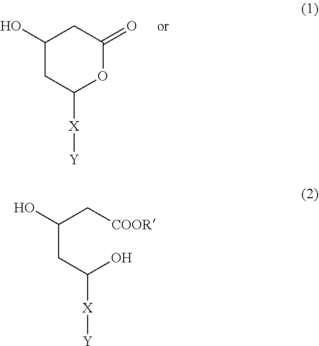

wherein X in each of formulas (1) and (2) represents a substituted or unsubstituted alkylene, alkenylene, or alkynylene linker of 2-6C;
Y represents one or more carbocyclic or heterocyclic rings; when Y comprises two or more rings, they may optionally be fused; and
R′ represents a cation, H or a substituted or unsubstituted alkyl group of 1-6C. It is understood that if R′ represents a cation with multiple positive charges, the appropriate number of anions is coupled with it. Formulas (1) and (2) are, respectively, the unhydrolyzed and hydrolyzed forms of the statin compounds. Preferred substituents on X (or on R′ when R′ is alkyl) are hydroxy, alkoxy, phenyl, amino and alkyl- or dialkylamino.

The compounds useful in the invention contain at least one and generally several chiral centers. Compounds useful in the invention include mixtures of the various stereoisomers and the stereoisomeric forms of the compounds individually. Preferred stereoisomers with respect to the compound of formula (1) are of the formula:

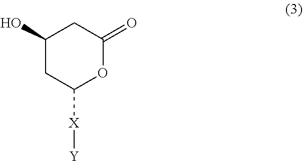

and the corresponding stereochemistry in the open chain (nonlactone or hydrolyzed) form of formula (2).

In one set of preferred embodiments, X is unsubstituted; most preferably X is selected from the group consisting of —CH2CH2-; —CH—CH—; and —C≡C—, especially-CH2CH2- and —CH═CH—.

Preferred embodiments of Y comprise ring systems such as naphthyl, polyhydro-naphthyl, monohydro- or dihydrophenyl, quinolyl, pyridyl, quinazolyl, pteridyl, pyrolyl, oxazoyl and the like and the reduced or partially reduced forms thereof.

Any of the embodiments of the invention may employ wherein the substituent Y includes those of the formula:

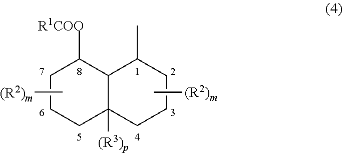

wherein the ring system may contain n-bonds;
wherein R1 is substituted or unsubstituted alkyl;
each R2 is independently a noninterfering substituent;
R3 is H, hydroxy, or alkoxy (1-6C);
each m is independently an integer of 0-6, wherein each R2 may reside in any of positions 2-7; and
p is 0 or 1, depending on the position of any π-bonds.

Particularly preferred embodiments include those of formulas (4a)-(4f) wherein the upper limit of n is adjusted according to the valence requirements appropriate for the particular ring system.

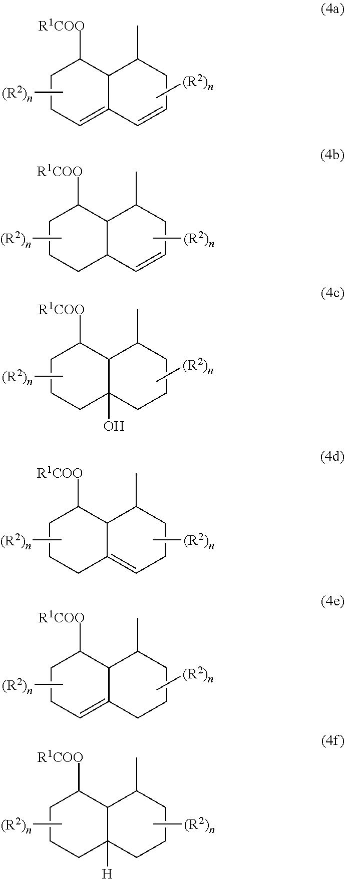

While R1 may be substituted alkyl, wherein the substituents may include hydroxy, alkoxy, alkylthiol, phenyl, phenylalkyl, and halo, unsubstituted alkyl is preferred. Particularly preferred embodiments of R1 are alkyl of 1-6C, including propyl, sec-butyl, t-butyl, n-butyl, isobutyl, pentyl, isopentyl, 1-methylbutyl, and 2-methylbutyl. Particularly preferred are propyl and sec-butyl.

Preferred embodiments for R2 include H, hydroxy, ═O, and substituted or unsubstituted lower alkyl (1-4C), in particular methyl, and hydroxymethyl. In the preferred embodiments, each n is independently 1 or 2 and preferred positions for substitution are positions 2 and 6 (see formula (4)). Particularly preferred embodiments of R2 are OH, H, and lower alkyl, in particular CH3.

Particularly preferred are embodiments wherein Y is 4 (a) or 4 (b), and especially embodiments having the substitution pattern indicated in formulas 4 (g) and 4 (h) below.

As indicated above, the compounds of the invention may be supplied as individual stereoisomers or as mixtures of stereoisomers. Preferred stereoisomers are those of the formulas (4g) and (4h) as typical and appropriate for those represented by the formulas (4a)-(4f).

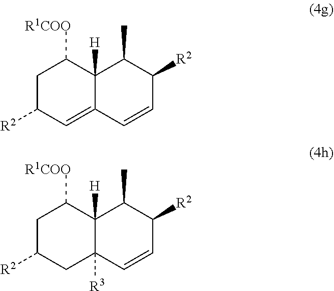

Particularly preferred are compounds with the stereochemistry of formulas (4g) and (4h) wherein the noted substituents are the sole substituents on the polyhydronaphthyl system optionally including additional substituents at position 5. Preferred embodiments include those wherein each of R2 is independently OH, CH2OH, methyl, or ═O. Preferred embodiments of R1 in these preferred forms are propyl, sec-butyl, and 2-methyl-but-2-yl.

Additional preferred embodiments of Y are:

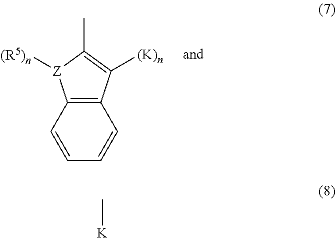

wherein Z is N and both n are 1, and each K comprises a substituted or unsubstituted aromatic or nonaromatic carbocyclic or heterocyclic ring system which may optionally be spaced from the linkage position shown in formula (7) by a linker of 1-2C, including μCHOH—, —CO—, and —CHNH2-, for example. Aromatic ring systems are preferred. Particularly preferred are compounds of formula (7), either as shown or wherein Z is contained in a 6-membered, rather than a 5-membered aromatic ring. Thus, another group of preferred compounds of the invention is of formula (7) where Z is N and an additional substituent —═CR6- is inserted between Z and the bond directed to X, wherein R6 is linear, branched or cyclic alkyl. In a preferred embodiment, R6, is a cyclic alkyl substituent.

R5 is H or linear, branched, cyclic substituted or unsubstituted alkyl, wherein substituents are preferably hydroxy, alkoxy, phenyl, amino and alkyl- or dialkylamino. Preferably, when R5 is alkyl, it is unsubstituted.

The substituents on the aromatic ring systems or nonaromatic ring systems of the invention including those designated by K can be any noninterfering substituents. Generally, the non-interfering substituents can be of wide variety. Among substituents that do not interfere with the beneficial effect of the compounds of the invention on bone formation in treated subjects include alkyl (1-6C, preferably lower alkyl 1-4C), including straight, branched or cyclic forms thereof, alkenyl (2-6C, preferably 2-4C), alkynyl (2-6C, preferably 2-4C), all of which can be straight or branched chains and may contain further substituents; halogens, including F, Cl, Br and I; silyloxy, OR, SR, NR2, OOCR, COOR, NCOR, NCOOR, and benzoyl, CF3, OCF3, SCF3, N (CF3) 2, CN, SO, SO2R and SO3R wherein R is alkyl (1-6C) or is H. Where two substituents are in adjacent positions in the aromatic or nonaromatic system, they may form a ring. Further, rings not fused to the aromatic or nonaromatic system K may be included as substituents. These rings may be aromatic and may be substituted or unsubstituted.

Preferred non-interfering substituents include hydrocarbyl groups of 1-6C, including saturated and unsaturated, linear or branched hydrocarbyl as well as hydrocarbyl groups containing ring systems; halo groups, alkoxy, hydroxy, CN, CF₃, and COOR, amino, monoalkyl- and dialkylamino where the alkyl groups are 1-6C. Particularly preferred are substituted or unsubstituted aromatic rings.

Although the number of substituents on a ring symbolized by K may typically be 0-4 or 0-5 depending on the available positions, preferred embodiments include those wherein the number on a single ring is 0, 1 or 2, preferably 0 or 1. However, an exception is that of formula (8), where it is preferred that the aromatic carbocyclic or heterocyclic ring system be multiply substituted. In particular, it is preferred that the substituents on K in formula (8) themselves contain aromatic rings. Particularly preferred are substituents that contain phenyl rings.

Particularly preferred are the embodiments of formula (7) or the ring expanded form thereof wherein K represents optionally substituted phenyl. Particularly preferred are compounds wherein R5 is isopropyl and K is para fluorophenyl forms.

Any of the embodiments of the invention may employ hydrolyzed or unhydrolyzed forms of lovastatin, mevastatin, simvastatin, fluvastatin, pravastatin, cerivastatin, NK-104 and atorvastatin.

Any of the embodiments of the invention may employ bisphosphonates and their analogs. Typically, and preferably, the bisphosphonates are of the formula

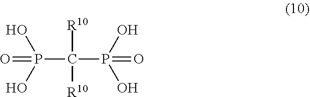

and the pharmaceutically acceptable salts, esters and amides thereof. Typical salts are those of the inorganic ions, such as sodium ion, potassium ion, calcium ion, magnesium ion and the like; any pharmaceutically acceptable cation may be used. Typical esters are the ethyl, methyl, isobutyl, ethylene glycol, and other typical pharmaceutically acceptable esters; typical amides are the unsubstituted —NH2 amides as well as the alkyl and dialkyl amides.

Embodiments of R10 include halo, OR, SR, NR2, where R is H or alkyl (1-6C) or alkyl or arylalkyl with optional substitutions. Particularly preferred are the amino-substituted alkyl embodiments. Typically, both R10 are not identical, although in some embodiments, such as clodronate, both R10 are halo. Particularly preferred compounds among the bisphosphonates are risedronate, alandronate, parnidronate, clodronate and in particular ibandronate. These compounds are particularly useful in combination with the statins.

The term “pharmaceutically acceptable” means compatible with the treatment of patients.

The term “solvate” as used herein means a statin, or a salt of a statin, wherein molecules of a suitable solvent are incorporated in the crystal lattice. A suitable solvent is physiologically tolerable at the dosage administered. The formation of solvates of statins will vary depending on the statin and the solvate. In general, solvates are formed by dissolving a compound in the appropriate solvent and isolating the solvate by cooling or using an antisolvent.

The solvate is typically dried or azeotroped under ambient conditions.

The term “pharmaceutically acceptable salt” includes both pharmaceutically acceptable acid addition salts and base addition salts.

The term “pharmaceutically acceptable acid addition salt” as used herein means any non-toxic organic or inorganic salt of any basic statin. Basic statins that may form an acid addition salt include, for example, those substituted with NH2, NHCI-Czoalkyl or N (C1-C2oalkyl) (CI-C2oalkyl). Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of statins are more soluble in water and various hydrophilic organic solvents, and generally demonstrate higher melting points in comparison to their free base forms. The selection of the appropriate salt will be known to one skilled in the art. Other non-pharmaceutically acceptable acid addition salts, e.g. oxalates, may be used, for example, in the isolation of the statins, for laboratory use, or for subsequent conversion to a pharmaceutically acceptable acid addition salt.

The term “pharmaceutically acceptable basic addition salt” as used herein means any non-toxic organic or inorganic base addition salt of any acid statin compound. Acidic statins that may form a basic addition salt include, for example, those possessing a carboxylic acid moiety. Illustrative inorganic bases which form suitable salts include lithium, sodium, potassium, calcium, magnesium or barium 1 o hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art. Other non-pharmaceutically acceptable basic addition salts, may be used, for example, in the isolation of the statins, for laboratory use, or for subsequent conversion to a pharmaceutically acceptable basic addition salt.

The formation of a desired compound salt is achieved using standard techniques. For example, the neutral statin is treated with an acid or base in a suitable solvent and the formed salt is isolated by filtration, extraction or any other suitable method.

The term “prodrug” as used herein refers to any compound that has less intrinsic activity than the corresponding “drug,” but when administered to a biological system, generates the “drug” substance, either as a result of spontaneous chemical reaction or by enzyme catalyzed or metabolic reaction. Prodrugs include, without limitation, acyl esters, carbonates, phosphates, and urethanes. These groups are exemplary, and not exhaustive, and one skilled in the art could prepare other known varieties of prodrugs. Prodrugs may be, for example, formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C1-C24) esters, acyloxymethyl esters, carbamates and amino acid esters.

Compositions used in method embodiments of the present technology are pharmaceutically formulated for administration via topical administration. Such formulations, besides containing a statin, may comprise appropriate salts, buffers, solvents, dispersion media, antibacterial and antifungal agents, isotonic agents, and absorption delaying agents to render delivery of the composition in a stable manner and thus allow uptake by target tissues (e.g., epidermal tissue). Supplementary active ingredients may also be incorporated into the compositions. In certain embodiments, administration is localized to a lesion or proximal to a psoriatic lesion or site.

Any of the embodiments of the invention may additionally contain therapeutically effective amounts of one or more compound which are known to be of use in the topical treatment of psoriasis and atopic dermatitis. These compounds are well known to those skilled in the art and include cyclosporine, methotrexate, tamoxifen, forskolin and analogs, tar derivatives, steroids, vitamin A and its derivatives vitamin D and its derivatives including 1-alpha-hydroxy-cholecalciferol, 1,25-dihydroxy-cholecalciferol, 24,25-dihydroxy-cholecalciferol, 1,24-dihydroxy-cholecalciferol and calcipotriol (MC 903), and beta agonists such as terbutaline.

Any of the embodiments of the invention may additionally employ an effective amount of at least one member of the group of adenosine and related purines, lipooxygenase inhibitors, substance P antagonists, delta tocopherol, 2-heptanone, and fatty acids and their esters such as heptanoic acid, ethyl heptanoate, 3,3-dimethylbutyric acid, and lipoic acid.

The term “dosage form” refers to topical or transdermal administration of statins used in the present technology and includes powders, sprays, ointments, pastes, creams, lotions, gels, solutions, films, foams, and transdermal patches. The active component may be mixed under sterile conditions with a pharmaceutically-acceptable carrier or excipient, and with any preservatives or buffers that may be important. Powders and sprays can be prepared, for example, with excipients such as lactose, talc, silicic acid, aluminum hydroxide, calcium silicates, and polyamide powder, or mixtures of these substances. The ointments, pastes, creams, and gels may also contain excipients such as animal and vegetable fats, oils, waxes, paraffins, starch, tragacanth, mixtures of monoacylglycerols, propylene glycol, oleic acid, ethoxyethoxy ethanol, cellulose derivatives, polyethylene glycols, silicones, bentonites, silicic acid, talc and zinc oxide, or mixtures thereof.

The term “transdermal penetration” refers to topical compositions formulated to optimize transport of an active from the skin surface into epidermal and dermal layers of the skin.

The term “Transcutol” refers to transcutolylene glycol monoethyl ether, 2-(2-ethoxyethoxy) ethanol and is a non-ionic surfactant and dissolvent with powerful solubilizing ability. It is used to increase the permeability of the drugs, and is listed in the FDA Inactive Ingredient Database for topical use.

The term “Capmul MCM EP” refers to Mono/diglycerides of capric acid and is an emulsifier and natural lipophilic surfactant enhancer, and dissolves hydrophobic substances with good solubilizing ability. In addition, it improves permeation and is also listed in the FDA Inactive Ingredient Database.

The phrases an “effective amount” or a “therapeutically effective amount” of a statin or ingredient, or pharmaceutically active agent or ingredient, which are synonymous herein, refer to an amount of the pharmaceutically active agent sufficient enough to have a positive effect on the patient. Accordingly, these amounts are sufficient to modify the disorder, condition, or appearance to be treated but low enough to avoid serious side effects, within the scope of sound medical advice.

A therapeutically effective amount of the pharmaceutically active agent will cause a substantial relief of symptoms when applied repeatedly over time. Effective amounts of the pharmaceutically active agent will vary with the particular condition or conditions being treated, the severity of the condition, the duration of the treatment, the specific components of the composition being used, and like factors.

### Liposome-Peg Statin-Micelles

Any of the embodiments of the invention may employ a liposomal-PEG composition including a statin incorporated into a micelle, the micelle is then combined with a liposome, such as a pre-formed liposome, thus incorporating the statin into the liposome.

In some embodiments, the statin may be solubilized in a solvent, such as ethanol or hydroalcoholic solutions of ethanol between about 1 to about 30% (v/v).

Any of the embodiments of the invention may employ a statin incorporated into a micelle. The statin is dissolved in, for example, aqueous buffer/alcoholic media and combined with a buffer solution comprising the micelle forming compound and the resulting combination mixed and warmed to a temperature of about 30 C to about 70 C, suitably about 55 C, until a substantially clear solution is obtained.

The statin-containing micelles are then incorporated into the liposome-PEG composition. Combining the solution of the statin-containing micelles with a buffered solution containing the preformed liposomes and warming to a temperature of about 30 C to about 70 C, suitably about 35 C to about 55 C until the statin-micelles are incorporated into the lipid bilayer of the liposome-PEG compositions.

Liposomal compositions according to the invention may be stored in any suitable form that may vary according to mode of administration. For example, a liposomal composition may be a liquid at room temperature (e.g., a sterile single-vial liquid), a frozen product, or a dehydrated product (e.g., a powder or a lyophilized cake to be reconstituted prior to use). Different storage forms may be prepared using methods known to a person skilled in the art. For example, a cryoprotectant such as a disaccharide, may be added to a liposomal composition prior to lyophilization to enable storage of a liposomal composition as a dehydrated product.

### EXAMPLES

Any of the embodiments of the invention may employ liposomal-PEG simvastatin prepared by a standard thin-film extrusion method (TFE) and the micelle-loading method (ML).

Any of the embodiments of the invention may employ simvastatin PEG-lipid micelles added to pre-formed liposomes having DMPC or DPPC. Simvastatin is rapidly loaded, and remains stably incorporated, into the outer layer of the liposomes.

Lipid composition is indicated with molar ratios of components indicated: dipalmitoyl phosphatidylcholine (DPPC); distearoyl phosphatidyl ethanolamine-poly (ethylene glycol) (DSPE-PEG); 30-[N-(Dimethyl-aminoethane)-carbamoyl] cholesterol (DC-chol); dimyristroyl phosphatidylcholine (DMPC:); dimyristoyl phosphatidyl glycerol (DMPG); simvastatin in lactone or carboxylic acid form (“activated simvastatin” “—COOH” using base (NaOH) in alcohol (EtOH). Prophetic examples provided below.

### TFE Examples

| Ingred. | molar ratio | molar ratio | molar ratio | molar ratio |
|---|---|---|---|---|
| DPPC | 90 | 85 | 90 | 85 |
| DC-Chol | 5 | 10 | 5 | 10 |
| DSPE-PEG | 5 | 5 | 5 | 5 |
| Simvastatin | 1.5 mg/ml | 2.8 mg/ml | 1.8 mg/ml | 2.2 mg/ml |

### Micelle Examples

| Ingred. | molar ratio | molar ratio | molar ratio | molar ratio |
|---|---|---|---|---|
| DPPC | 90 | 85 | 90 | 85 |
| DC-Chol | 5 | 10 | 5 | 10 |
| DSPE-PEG | 5 | 5 | 5 | 5 |
| Simvastatin | 1.0 mg/ml | 1.4 mg/ml | 1.3 mg/ml | 1.5 mg/ml |

### TFE Example

| Ingred. | molar ratio |
|---|---|
| DMPG | 67 |
| DMPC | 28 |
| PEG | 5 |
| Simvastatin | 0.6 mg/ml |

### Micelle Example

| Ingred. | molar ratio |
|---|---|
| DMPG | 67 |
| DMPC | 28 |
| PEG | 5 |
| Simvastatin | 1.7 mg/ml |

### THE Examples

| Ingred. | molar ratio | molar ratio | molar ratio | molar ratio |
|---|---|---|---|---|
| DPPC | 90 | 85 | 90 | 85 |
| DC-Chol | 5 | 10 | 5 | 10 |
| DSPE-PEG | 5 | 5 | 5 | 5 |
| Simvastatin- | 1.0 mg/ml | 1.3 mg/ml | 1.0 mg/ml | 1.0 mg/ml |
| COOH | | | | |

### Micelle Examples

| Ingred. | molar ratio | molar ratio | molar ratio | molar ratio |
|---|---|---|---|---|
| DPPC | 90 | 85 | 90 | 85 |
| DC-Chol | 5 | 10 | 5 | 10 |
| DSPE-PEG | 5 | 5 | 5 | 5 |
| Simvastatin- | 1.5 mg/ml | 2.0 mg/ml | 1.5 mg/ml | 1.6 mg/ml |
| COOH | | | | |

### Dosage Forms

Dosage formulations for topical administration include, but are not limited to, ointments, creams, emulsions, lotions, gels, sunscreens and agents that favor penetration within the epidermis.

Any of the embodiments of the invention may employ additives including solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers.

Any of the embodiments of the invention may employ skin permeation enhancers including, without limitation lower alkanols, such as methanol ethanol and 2-propanol; alkyl methyl sulfoxides such as dimethylsulfoxide (DMSO), decylmethylsulfoxide (C10 MSO) and tetradecylmethyl sulfoxide; pyrrolidones, urea; N,N-diethyl-m-toluamide; C2-C6 alkanediols; dimethyl formamide (DMF), N,N-dimethylacetamide (DMA) and tetrahydrofurfuryl alcohol.

Any of the embodiments of the invention may employ solubilizers including, but are not limited to, hydrophilic ethers such as diethylene glycol monoethyl ether (ethoxydiglycol, available commercially as Transcutol®) and diethylene glycol monoethyl ether oleate (available commercially as Softcutol®); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol®); alkyl methyl sulfoxides, such as DMSO; pyrrolidones, DMA, and mixtures thereof.

Any of the embodiments of the invention may employ a lipoprotein, such as apolipoprotein E (ApoE). In some embodiments, the composition comprises a lipoprotein and a statin that is conjugated to a lipophilic, eg, cholesterol. ApoE is an at least one amphiphile such as phospholipids such as dimyristoyl phosphatidylcholine (DMPC), dioleoylphosphatidylethanolamine (DOPE), palmitoyl oleoyl phosphatidylcholine (POPC), egg phosphatidylcholine (EPC), Stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), and dioleoyl-phosphatidylethanol Amine 4-(N-maleimidomethyl)-cyclohexane-1-carboxylate DOPE-mal).

### Liposomal Formulations

Any of the embodiments of the invention may employ liposomes and liposome-PEG compositions and include monolayers, micelles, bilayers and vesicles. In some embodiments, the term “liposome” means a vesicle composed of amphiphilic lipids arranged in a spherical bilayer or bilayers.

In one non-limiting embodiment, the lipid particle comprises 45-65 mol % of a cationic lipid, 5 mol % to about 10 mol %, of a non-cationic lipid, 25-40 mol % of a sterol, and 0.5-5 mol % of a PEG or PEG-modified lipid.

Liposomes are unilamellar or multilamellar vesicles which have a membrane formed from a lipophilic material and an interior. The interior portion contains the composition to be delivered. Cationic liposomes possess the advantage of being able to fuse to the cell wall. Non-cationic liposomes, although not able to fuse as efficiently with the cell wall, are taken up by macrophages in vivo.

Liposomes and liposome-PEG are useful for the transfer and delivery of statins to the skin. Because the liposomal membrane is structurally similar to biological membranes, when liposomes are applied to a tissue, the liposomes start to merge with the tissue and as the merging of the liposome and tissue progresses, the contents are delivered where the statin may act.

Any of the embodiments of the invention may employ phospholipids including dimyristoyl phosphatidylcholine (DMPC) or dipalmitoyl phosphatidylcholine (DPPC), dimyristoyl phosphatidylglycerol, dioleoyl phosphatidylethanolamine (DOPE), phosphatidylcholine (PC) and mixtures of phospholipid and/or phosphatidylcholine and/or cholesterol.

Any of the embodiments of the invention may employ “sterically stabilized” liposomes including glycolipids, including monosialoganglioside GM1, galactocerebroside sulfate, phosphatidylinositol, sphingomyelin, the ganglioside GM1, a galactocerebroside sulfate ester, and 1,2-sn-dimyristoylphosphat-idylcholine.

Any of the embodiments of the invention may employ a lipid derivatized with one or more hydrophilic polymers, including liposomes comprising a nonionic detergent 2C1215G, liposomes that contains a PEG moiety, liposomes having a hydrophilic coating of polymeric glycol on polystyrene particle, synthetic phospholipids modified by the attachment of carboxylic groups of polyalkylene glycols (e.g., PEG), liposomes comprising phosphatidylethanolamine (PE) derivatized with PEG or PEG stearate, PEG-derivatized phospholipids, e.g., DSPE-PEG, formed from the combination of distearoylphosphatidylethanolamine (DSPE) and PEG, liposomes having covalently bound PEG moieties on their external surface, liposome compositions containing 1-20 mole percent of PE derivatized with PEG, liposomes comprising PEG-modified ceramide lipids, and PEG-containing liposomes further derivatized with functional moieties on their surfaces.

Liposomal-PEG particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic

In one embodiment, the lipid to statin ratio (mass/mass ratio) (e.g., lipid to statin ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or about 6:1, 7:1, 8:1, 9:1, 10:1, 11:1, 12:1, or 33:1.

### Cationic Lipids

Any of the embodiments of the invention may employ a cationic lipid. The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta [d][1,3]dioxol-5-amine (ALNY-100), (6Z,9Z,28Z,31Z)-heptatriaconta-6,9,28,31-tetraen-19-yl 4-(dimethylamino) butanoate (MC3), or a mixture thereof.

Additional cationic lipids include, but are not limited to, N,N-dioleyl-N,N-dimethylammonium chloride (“DODAC”); N-(2,3-dioleyloxy) propyl-N,N—N-triethylammonium chloride (“DOTMA”); N,N-distearyl-N,N-dimethylammonium bromide (“DDAB”); N-(2,3-dioleoyloxy) propyl)-N,N,N-trimethylammonium chloride (“DOTAP”); 1,2-Dioleyloxy-3-trimethylaminopropane chloride salt (“DOTAP.Cl”); 3β-(N—(N′,N′-dimethylaminoethane)-carbamoyl) cholesterol (“DC-Chol”), N-(1-(2,3-dioleyloxy) propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy) propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). In particular embodiments, a cationic lipid is an amino lipid.

### Non-Cationic Lipids

Any of the embodiments of the invention may employ a non-cationic lipid including but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof.

### Anionic Lipids

Any of the embodiments may include anionic lipids include phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

### Neutral Lipids

Any of the embodiments may include neutral lipids, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C14 to C22 are preferred. In another group of embodiments, lipids with mono- or di-unsaturated fatty acids with carbon chain lengths in the range of C14 to C22 are used. Additionally, lipids having mixtures of saturated and unsaturated fatty acid chains can be used. Preferably, the neutral lipids used in the invention are DOPE, DSPC, POPC, or any related phosphatidylcholine. The neutral lipids useful in the invention may also be composed of sphingomyelin, dihydrosphingomyeline, or phospholipids with other head groups, such as serine and inositol.

Any of the embodiments may include a non-cationic lipid is distearoylphosphatidylcholine (DSPC). In another embodiment the non-cationic lipid is dipalmitoylphosphatidylcholine (DPPC).

The non-cationic lipid may be from about 5 mol % to about 90 mol %, about 5 mol % to about 10 mol %, about 10 mol %, or about 58 mol % if cholesterol is included, of the total lipid present in the particle.

### Conjugated Lipids

Any of the embodiments may include conjugated lipids, including polyethylene glycol (PEG)-modified lipids, monosialoganglioside Gm1, and polyamide oligomers (“PAO”). Typically, the concentration of the lipid component is about 1 to 15% (by mole percent of lipids).

Specific examples of PEG-modified lipids (or lipid-polyoxyethylene conjugates) that are useful in the invention can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines Particularly preferred are PEG-modified diacylglycerols and dialkylglycerols.

Other conjugates, such as PEG-CerC20 have similar staying capacity. At least three characteristics influence the rate of exchange: length of acyl chain, saturation of acyl chain, and size of the steric-barrier head group. Compounds having suitable variations of these features may be useful for the invention. For some therapeutic applications, it may be preferable for the PEG-modified lipid to be rapidly lost from the statin-lipid particle in vivo and hence the PEG-modified lipid will possess relatively short lipid anchors. In other therapeutic applications, it may be preferable for the statin-lipid particle to exhibit a longer plasma circulation lifetime and hence the PEG-modified lipid will possess relatively longer lipid anchors. Exemplary lipid anchors include those having lengths of from about C14 to about C22, preferably from about C14 to about C16. In some embodiments, a PEG moiety, for example an mPEG-NH2, has a size of about 1000, 2000, 5000, 10,000, 15,000 or 20,000 Daltons.

Any of the embodiments may include, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci2), a PEG-dimyristyloxypropyl (Ci4), a PEG-dipalmityloxypropyl (Ci6), or a PEG-distearyloxypropyl (C]8). Additional conjugated lipids include polyethylene glycol-didimyristoyl glycerol (C14-PEG or PEG-C14, where PEG has an average molecular weight of 2000 Da) (PEG-DMG); (R)-2,3-bis (octadecyloxy) propyl1-(methoxy poly (ethylene glycol) 2000) propylcarbamate) (PEG-DSG); PEG-carbamoyl-1,2-dimyristyloxypropylamine, in which PEG has an average molecular weight of 2000 Da (PEG-cDMA); N-Acetylgalactosamine-((R)-2,3-bis (octadecyloxy) propyl1-(methoxy poly (ethylene glycol) 2000) propylcarbamate)) (GalNAc-PEG-DSG); and polyethylene glycol-dipalmitoylglycerol (PEG-DPG).

In one embodiment the conjugated lipid is PEG-DMG. In another embodiment the conjugated lipid is PEG-CDMA. In still another embodiment the conjugated lipid is PEG-DPG. Alternatively the conjugated lipid is GalNAc-PEG-DSG.

The conjugated lipid may be from 0 mol % to about 20 mol % or about 0.5 to about 5.0 mol % or about 2 mol % of the total lipid present in the particle.

The sterol component of the lipid mixture, when present, can be any of those sterols conventionally used in the field of liposome, lipid vesicle or lipid particle preparation. A preferred sterol is cholesterol.

Any of the embodiments may comprise a statin-lipid particle that further includes a sterol, e.g., a cholesterol at, e.g., about 10 mol % to about 60 mol % or about 25 to about 40 mol % or about 48 mol % of the total lipid present in the particle.

### Amphipathic Lipids

Any of the embodiments may include amphipathic lipids, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

### Layers

Any of the embodiments may include the statin encapsulated within an aqueous interior of the lipid particle. In other embodiments, the statin is present within one or more lipid layers of the lipid particle. In other embodiments, the active agent is bound to the exterior or interior lipid surface of a lipid particle.

### Penetration Enhancers

Any of the embodiments of the invention may employ various penetration enhancers to effect the efficient delivery of statins to the skin of animals. Transdermal penetration with a statin for topical application requires both high solubility and high permeability or penetration with molecular weight less than 500 Daltons. The stratum corneum is hydrophobic and a barrier for any topically applied reagent. Lovastatin has limited solubility, atorvastatin has relative heavier molecular weight (604 Da) and so penetration is limited. Simvastatin is a hydrophobic statin with a moderate molecular weight (418 Da) and potentially increased penetration capability, however the liposomal-PEG formulation is required for effective delivery.

Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants.

Surfactant penetration enhancers include, for example, sodium lauryl sulfate, polyoxyethylene-9-lauryl ether and polyoxyethylene-20-cetyl ether).

Fatty acids penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C1-10 alkyl esters thereof (e.g., methyl, isopropyl and t-butyl), and mono- and di-glycerides thereof (i.e., oleate, laurate, caprate, myristate, palmitate, stearate, linoleate, etc.)

Bile salts penetration enhancers include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE).

Chelating agent penetration enhancers include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines).

### Lipid Vesicles Containing Hyaluronic Acid and Optionally Collagen

Any of the embodiments may include hyaluronic acid incorporated into lipid vesicles to deliver hyaluronic acid to a patient's skin. As described herein, any lipid vesicle suitable for encapsulating hyaluronic acid and for administration to the skin of a human, provided it is a non-phospholipid paucilaminar liposome.

Any of the embodiments may include lipid vesicles incorporating hyaluronic acid and optionally human collagen (and/or a fragment thereof) or a collagen derivative. Vesicles containing the active agent(s) are useful for administering the active agent(s) to a subject. As lipid vesicle, a non-phospholipid paucilaminar liposome is used.

The vesicles of the invention are vesicles having one or more lipid bilayer membranes surrounding a cavity. The lipid vesicles for use in the invention are in the range of about 50 to about 950 nm (eg, 50, 100, 200, 300, 400, 500, 600, 700, 800, 900, 950 nm) in size.

The collagen to be encapsulated in the lipid vesicles may be in any suitable form, for example, a type I collagen preparation, type III collagen, a mixture of type I collagen and type III collagen, a collagen derivative or a combination thereof.

The hyaluronic acid can be as described above. The vesicles can contain only HA or a combination of HA and collagen. The compositions of the invention may comprise vesicles containing only one active agent, or multiple active agents.

The lipid vesicles of the invention may include non-phospholipid “membrane mimic” amphiphiles that have a hydrophilic head group attached to a hydrophobic tail and include long-chain fatty acids, long-chain alcohols and derivatives, long-chain aminos and glycerolipids. In bilayers, the fatty acid tails point to the inside of the membrane and the polar-headed clusters point outward. Lipid-soluble molecule added during vesicle formation are incorporated into the nucleus of the vesicles.

Paucilamine vesicles that can be formed from many single-tailed, bio-compatible amphiphiles are used in the invention. Such paucilamellar lipid vesicles include non-phospholipid vesicles having one or more lipid bilayer membranes surrounding a large amorphous nucleus in which a chemical entity of interest (ie HA and optionally collagen) is encapsulated.

Any of the embodiments may include non-phospholipid paucilamellar lipid vesicles sold under the trade name Novasome® (IGI Inc., Buena, NJ). There are several formulations of Novasome® (for example, Novasome® A, Novasome® D, Novasome® Day Cream). Novasome® lipid vesicles are generally approximately 200-700 nanometers in size, depending on a wide variety of membrane constituents individually chosen for each particular purpose. Size distribution is uniform, and encapsulation efficiency can be almost 100% for lipid load. Finely divided insoluble particles (eg, insoluble pharmaceuticals) can also be encapsulated.

### Example: Cream Formulation

| Ingredient | % w/w |
|---|---|
| Stearic Acid | 15.0 |
| Cetyl Alcohol | 1.0 |
| White Petrolatum | 3.0 |
| Polyoxy 40 Stearate | 4.0 |
| Methyl Paraben | 0.025 |
| Propyl Paraben | 0.015 |
| LIPOSOME-PEG | 20-50 |
| simvastatin | 2.0-20.0 |
| Propylene Glycol | 9.5 |
| Sorbitol | 7.5 |

### Example: Transdermal Cream

| Ingredient | % w/w |
|---|---|
| LIPOSOME-PEG | 20-50 |
| simvastatin | 2.0-20.0 |
| Urea | 5.0 |
| Lactic Acid | 2.0 |
| Germaben II | 1.0 |
| Sorbitol 70 solution | 4.0 |
| Glycerin | 4.0 |
| White Petrolatum | 3.0 |
| Glyceryl monostearate | 3.0 |
| Cetyl alcohol | 3.5 |
| Heavy mineral oil | 10.0 |
| Eutanol G-Octyldodecanol | 5.0 |
| Acetylated lanolin (Modulan ™) | 3.0 |
| Myrg 52 ™ | 3.0 |
| Span 60 ™ | 1.5 |

Ammonia solution to adjust pH to 4.5

### Example: Transdermal Ointment

| Ingredient | % w/w |
|---|---|
| Miglyol ™ 840-B Gel | 10.0 |
| Eutanol G-Octyldodecanol | 17.0 |
| Cril 6-Glyceryl isostearate | 3.0 |
| Hard paraffin wax | 3.0 |
| Zinc stearate | 1.0 |
| Amphisol K | 0.5 |
| Germaben II | 1.0 |
| Magnesium sulfate | 0.2 |
| Urea | 10.0 |
| LIPOSOME-PEG | 20-50 |
| simvastatin | 2.0-20.0 |

### Example: Topical Gel

| Ingredient | % w/w |
|---|---|
| simvastatin | 2.0 |
| Carbopol 940 ™ | 1.5 |
| Triethanolamine | 1.5 |
| Water | 95.0. |

### Example—Cream

| ingredient | % W/W |
|---|---|
| simvastatin | 2.0-10.0 |
| liposome-PEG | 20-50 |
| Urea | 5.0 |
| Lactic acid | 2.0 |
| Ammonia | 1.0 |
| Germaben ii | 1.0 |
| Sorbitol 70% | 4.0 |
| Glycerol | 4.0 |
| White petrolatum | 3.0 |
| Glyeryl monostearate | 3.5 |
| Cetyl alcohol | 3.5 |
| Mineral oil | 10.0 |
| Octyldecanol | 5.0 |
| Acetylated lanolin | 3.0 |
| Polyoxyl 40 stearate | 3.0 |
| Sorbitan monostearate | 1.5 |

### Example—pH 3.8-4.5 Cream

| ingredient | % W/W |
|---|---|
| simvastatin | 1.0-5.0 |

Aqua, Glycerin, Cetearyl Alcohol, Caprylic/Capric Triglyceride, Cetyl Alcohol, Ceteareth-20, Petrolatum, Potassium Phosphate, Ceramide NP, Ceramide AP, Ceramide EOP, Carbomer, Dimethicone, Behentrimonium Methosulfate, Sodium Lauroyl Lactylate, Sodium Hyaluronate, Cholesterol, Phenoxyethanol, Disodium EDTA, Dipotassium Phosphate, Tocopherol, Phytosphingosine, Xanthan Gum, Ethylhexylglycerin.

pH: 3.8-4.5

### Example—pH 3.8-4.5 Cream

| ingredient | % W/W |
|---|---|
| simvastatin | 1.0-5.0 |
| liposome-PEG | 20-50 |

Aqua, Glycerin, Cetearyl Alcohol, Caprylic/Capric Triglyceride, Cetyl Alcohol, Ceteareth-20, Petrolatum, Potassium Phosphate, Ceramide NP, Ceramide AP, Ceramide EOP, Carbomer, Dimethicone, Behentrimonium Methosulfate, Sodium Lauroyl Lactylate, Sodium Hyaluronate, Cholesterol, Phenoxyethanol, Disodium EDTA, Dipotassium Phosphate, Tocopherol, Phytosphingosine, Xanthan Gum, Ethylhexylglycerin.

pH: 3.8-4.5

Referring now to the FIGURES.

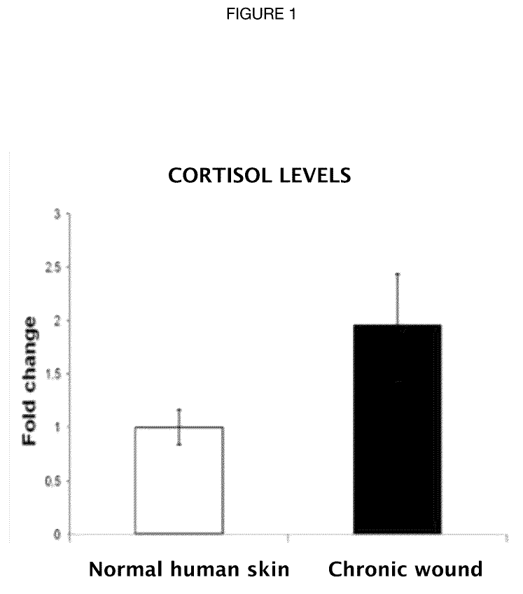

FIG. 1 shows that samples taken from patients with chronic diabetic wounds show that patients have elevated cortisol levels-almost twice the levels of healthy skin.

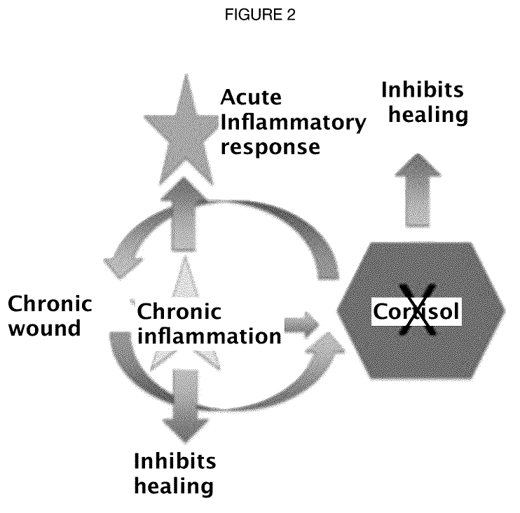

FIG. 2 shows that these levels result in a feedback loop that keeps a wound stuck in a chronic stage vs an acute stage.

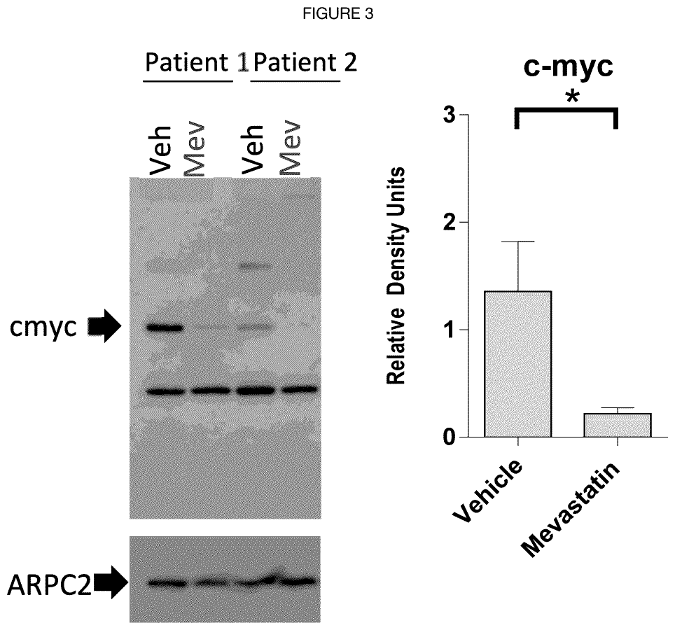

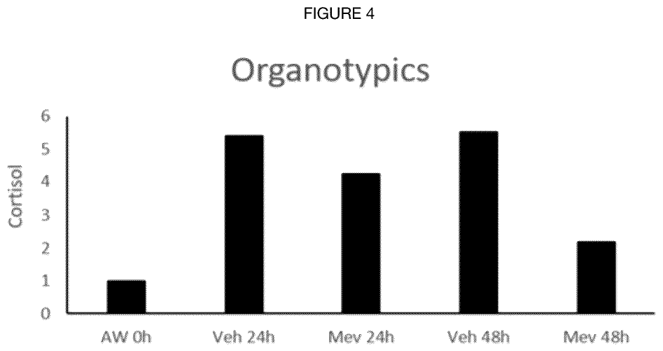

FIGS. 3 and 4 shows that statins are HMG-COA reductase inhibitors, of which cholesterol is a downstream target. Without cholesterol, local cortisol production should drop because cholesterol is required for cortisol synthesis. This was confirmed in primary human keratinocyte cultures and organotype wound healing models after treatment with mevastatin.

FIG. 3 shows c-myc expression in human skin ex-vivo and, FIG. 4, organotype wound models, treated with mevastatin. The results show c-myc levels drop drastically over a relatively short time frame. Samples are directly from chronic wounds, cultured, and measured c-myc levels before and after treatment with mevastatin. The results show a drop in c-myc levels. A large effect of high c-myc levels is that they cause abnormal differentiation and migration of keratinocytes. This results in a buildup of keratinocytes, indicated by the calloused margin at the end of the wound.

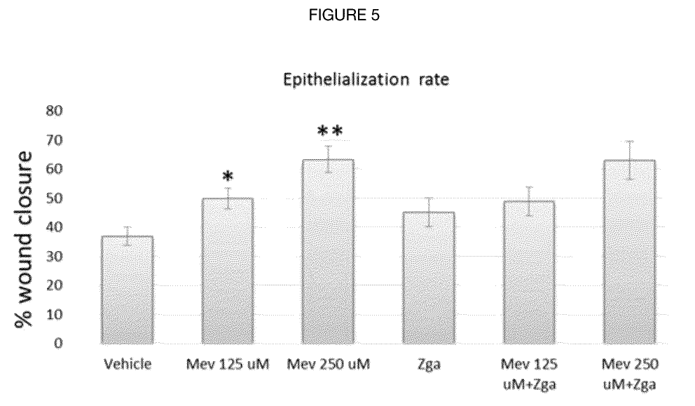

FIG. 5 shows that the drop in c-myc levels is commensurate with increased keratinocyte migration, which we tested via a scratch test. Primary human keratinocytes are used with varying doses of mevastatin and ZGA, and show increasing migration levels with dose. Measured epithelialization rates in partial thickness wounds at varying doses at 4 days, and confirms that statins can be used to aid wound healing.

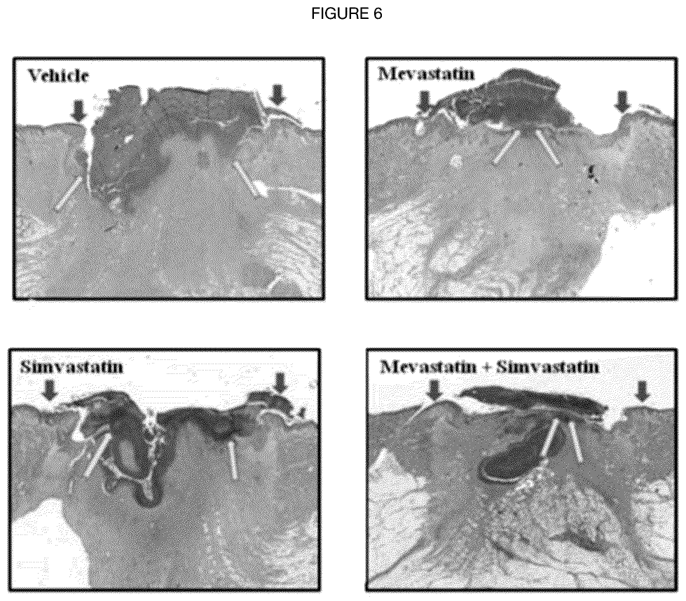

FIG. 6 shows simvastatin tested systemically in db/db mice enhances nitric oxide wound content, increase breaking strength, and promote angiogenesis. Topical simvastatin in mice shows significant acceleration of wound healing and an increase in angiogenesis. Mevastatin, simvastatin, and a combination were tested in a full thickness diabetic porcine model. FIG. 6 confirms that statins are an effective treatment of chronic wounds with a known mechanism of action. The compounds show increased epithelialization and vascularization.

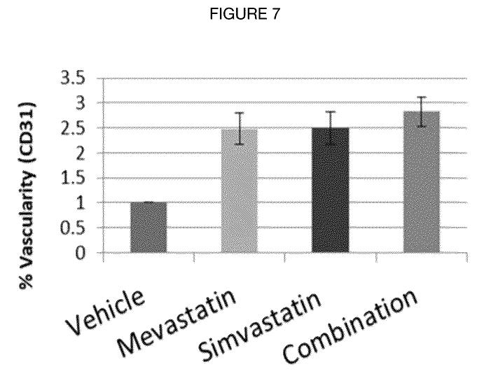

FIG. 7 shows % vascularity and a comparison of vehicle, mevastatin, simvastatin, and a combination of statins, and shows a nearly 3-fold increase in wound vascularity when treated with a statin or combination of statins.

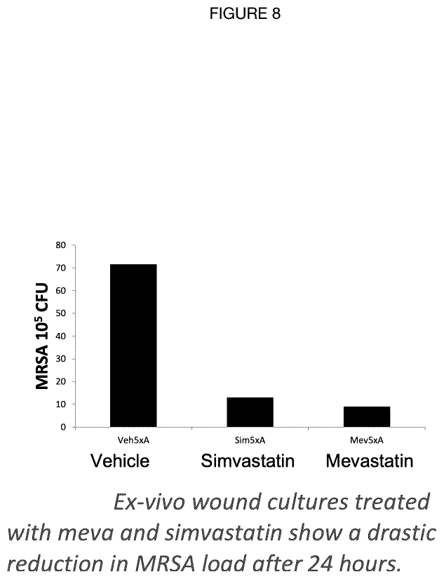

FIG. 8 shows biofilm disruption for methicillin resistant staph aureus (MRSA), and at 2-4×MIC, simvastatin transitioned from bacteriostatic to bactericidal, showing complete inhibition. Additionally, simvastatin has been shown to suppress multiple biosynthetic and cellular processes in bacteria around protein synthesis and the creation of key MRSA toxins.

The embodiments herein, and/or the various features or advantageous details thereof, are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. Rather, these embodiments are provided so that this disclosure will be thorough and complete, and will fully convey the scope of the inventive concepts to those skilled in the art. Like numbers refer to like elements throughout.

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where embodiments described above indicate certain components arranged in certain orientations or positions, the arrangement of components may be modified. While the embodiments have been particularly shown and described, it will be understood that various changes in form and details may be made. Any portion of the invention described herein may be combined in any combination, except mutually exclusive combinations.

The embodiments described herein can include various combinations and/or sub-combinations of the functions, components, and/or features of the different embodiments described. Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed:

1. A method for treating lichen sclerosus and/or psoriasis by administering to a patient in need thereof a topical pharmaceutical formulation comprising a statin 1.0%-5.0% w/w/in a non-aqueous, low pH, biphasic liposomal-PEG carrier.

2. The method according to claim 1, wherein the statin is simvastatin.

3. A topical pharmaceutical formulation comprising a statin 1.0%-5.0% w/w/in a non-aqueous, low pH, biphasic liposomal-PEG carrier.

4. The topical pharmaceutical formulation according to claim 3, wherein the statin is simvastatin.

5. The topical pharmaceutical formulation according to claim 4, wherein simvastatin is 2.5%-3.5% w/w.

6. The topical pharmaceutical formulation according to claim 4, wherein simvastatin is 3.5% w/W.

7. The method according to claim 1, wherein the statin is selected from the group consisting of simvastatin, cerivastatin, lovastatin, pravastatin, atorvastatin, fluvastatin, NK-104, and mixtures thereof.

8. The topical pharmaceutical formulation according to claim 6, comprising a penetration enhancer selected from the group consisting of urea, lactic acid, ammonium lactate, salicylic acid and a C3-C12-straight chain alkanoic acid.

9. The topical pharmaceutical formulation according to claim 8, wherein the pharmaceutical formulation is selected from the group consisting of a lotion, a cream, an ointment, a gel, a film, a foam, and a sprayable aerosol.

10. The topical pharmaceutical formulation according to claim 4, wherein said simvastatin is present at: i) a concentration of 45-150 μM of said topical pharmaceutical formulation, or ii) a concentration of 225-400 μM of said topical pharmaceutical formulation.

11. The topical pharmaceutical formulation according to claim 6, wherein the topical pharmaceutical formulation includes cholesterol, cholesterol derivative, or cholesterol analog is present at 2-10% in a pharmaceutically acceptable carrier.

12. The topical pharmaceutical formulation according to claim 11, wherein said cholesterol derivative is a cholesterol ester selected from the group consisting of: cholesteryl capronate, cholesteryl pelargonate, cholesteryl caprinate, cholesteryl undecilate, cholesteryl laurate, cholesteryl tridecilate, cholesteryl miristinate, cholesteryl palmitate, and cholesteryl stearate.

13. The topical pharmaceutical formulation according to claim 3, wherein said statin is selected from the group consisting of: simvastatin, rosuvastatin, lovastatin, cerivastatin, fluvastatin, mevastatin, pitavastatin, and pravastatin.

14. The topical pharmaceutical formulation according to claim 3, wherein said statin has molecular weight less than 500 daltons.

15. The topical pharmaceutical formulation according to claim 12, wherein said composition comprises an emulsion.

16. The topical pharmaceutical formulation according to claim 15, wherein said emulsion comprises an microemulsion, and wherein said microemulsion comprises an oil phase, a first surfactant, a second surfactant, and an oil phase.

17. The topical pharmaceutical formulation according to claim 15, wherein said emulsion comprises an oil-in-water emulsion.

18. The topical pharmaceutical formulation according to claim 15, wherein said emulsion comprises a water-in-oil emulsion.

19. The topical pharmaceutical formulation according to claim 15, wherein said emulsion comprises a surfactant and a solvent.

20. The topical pharmaceutical formulation according to claim 19, wherein said surfactant comprises monoacylglycerols.

21. The topical pharmaceutical formulation according to claim 19, where said solvent is selected from the group consisting of: water polyethylene glycol, oleic acid, and 2-(2-ethoxyethoxy) ethanol.

22. The method according to claim 2, wherein said low pH is pH from 3.8-4.5.

23. The topical pharmaceutical formulation according to claim 6, having a pH from 3.8-4.5.

24. The method according to claim 22, wherein the topical pharmaceutical formulation comprises a mixture of ceramides (˜40-50% by weight), hyaluronic acid (˜10-25% by weight), cholesterol (˜10-25% by weight), and free fatty acids (˜5-15% by weight).

25. The topical pharmaceutical formulation according to claim 23, comprising a mixture of ceramides (˜40-50% by weight), hyaluronic acid (˜10-25% by weight), cholesterol (˜10-25% by weight), and free fatty acids (˜5-15% by weight).

26. The method according to claim 24, wherein the topical pharmaceutical formulation comprises an equimolar mixture of ceramides (˜50% by weight), cholesterol (˜25% by weight), and free fatty acids (˜15% by weight).

27. The topical pharmaceutical formulation according to claim 25, comprising an equimolar mixture of ceramides (˜50% by weight), cholesterol (˜25% by weight), and free fatty acids (˜15% by weight).

28. The method according to claim 26, wherein the topical pharmaceutical formulation comprises a mixture of buffered glycolic acid and ammonia.

29. The topical pharmaceutical formulation according to claim 27, comprising a mixture of buffered glycolic acid and ammonia.

30. The method of claim 22, wherein the topical pharmaceutical formulation comprises a mixture of buffered glycolic acid and ammonia, in a formulation comprising aqua, isohexadecane, cetearyl isononanoate, dicaprylyl ether, sorbitan oleate, glycerin, hydrogenated vegetable oil, polyglyceryl-3 polyricinoleate, sucrose polystearate, magnesium sulfate, parfum, tocopherol, limonene, Helianthus annuus seed oil, linalool, BHT, citral.

31. The topical pharmaceutical formulation according to claim 23, comprising a mixture of buffered glycolic acid and ammonia, in a formulation comprising aqua, isohexadecane, cetearyl isononanoate, dicaprylyl ether, sorbitan oleate, glycerin, hydrogenated vegetable oil, polyglyceryl-3 polyricinoleate, sucrose polystearate, magnesium sulfate, parfum, tocopherol, limonene, Helianthus annuus seed oil, linalool, BHT, citral.

32. The method of claim 22, wherein the topical pharmaceutical formulation comprises a mixture of ceramides (˜40-50% by weight), hyaluronic acid (˜10-25% by weight), cholesterol (˜10-25% by weight), and free fatty acids (˜5-15% by weight), with buffered glycolic acid and ammonia, in a formulation comprising aqua, isohexadecane, cetearyl isononanoate, dicaprylyl ether, sorbitan oleate, glycerin, hydrogenated vegetable oil, polyglyceryl-3 polyricinoleate, sucrose polystearate, magnesium sulfate, parfum, tocopherol, limonene, Helianthus annuus seed oil, linalool, BHT, citral.

33. The topical pharmaceutical formulation according to claim 23, comprising a mixture of ceramides (˜40-50% by weight), hyaluronic acid (˜10-25% by weight), cholesterol (˜10-25% by weight), and free fatty acids (˜5-15% by weight), with buffered glycolic acid and ammonia, in a formulation comprising aqua, isohexadecane, cetearyl isononanoate, dicaprylyl ether, sorbitan oleate, glycerin, hydrogenated vegetable oil, polyglyceryl-3 polyricinoleate, sucrose polystearate, magnesium sulfate, parfum, tocopherol, limonene, Helianthus annuus seed oil, linalool, BHT, citral.

34. The method of claim 22, wherein the topical pharmaceutical formulation comprises a pH 3.8-4.5 cream having simvastatin 1.0-5.0% w/w, in a formulation comprising Aqua, Glycerin, Cetearyl Alcohol, Caprylic/Capric Triglyceride, Cetyl alcohol, Ceteareth-20, Petrolatum, Potassium Phosphate, Ceramide NP, Ceramide AP, Ceramide EOP, Carbomer, Dimethicone, Behentrimonium Methosulfate, Sodium Lauroyl Lactylate, Sodium Hyaluronate, Cholesterol, Phenoxyethanol, Disodium EDTA, Dipotassium Phosphate, Tocopherol, Phytosphingosine, Xanthan Gum, and Ethylhexylglycerin.

35. The method of claim 22, wherein the topical pharmaceutical formulation comprises a pH 3.8-4.5 cream having simvastatin 1.0-5.0% w/w, in liposome-PEG 20-50%, in a formulation comprising Aqua, Glycerin, Cetearyl Alcohol, Caprylic/Capric Triglyceride, Cetyl Alcohol, Ceteareth-20, Petrolatum, Potassium Phosphate, Ceramide NP, Ceramide AP, Ceramide EOP, Carbomer, Dimethicone, Behentrimonium Methosulfate, Sodium Lauroyl Lactylate, Sodium Hyaluronate, Cholesterol, Phenoxyethanol, Disodium EDTA, Dipotassium Phosphate, Tocopherol, Phytosphingosine, Xanthan Gum, and Ethylhexylglycerin.

36. The topical pharmaceutical formulation according to claim 23, comprising a pH 3.8-4.5 cream having simvastatin 1.0-5.0% w/w, in a formulation comprising Aqua, Glycerin, Cetearyl Alcohol, Caprylic/Capric Triglyceride, Cetyl alcohol, Ceteareth-20, Petrolatum, Potassium Phosphate, Ceramide NP, Ceramide AP, Ceramide EOP, Carbomer, Dimethicone, Behentrimonium Methosulfate, Sodium Lauroyl Lactylate, Sodium Hyaluronate, Cholesterol, Phenoxyethanol, Disodium EDTA, Dipotassium Phosphate, Tocopherol, Phytosphingosine, Xanthan Gum, and Ethylhexylglycerin.

37. The topical pharmaceutical formulation according to claim 23, comprising a pH 3.8-4.5 cream having simvastatin 1.0-5.0% w/w, in liposome-PEG 20-50%, in a formulation comprising Aqua, Glycerin, Cetearyl Alcohol, Caprylic/Capric Triglyceride, Cetyl Alcohol, Ceteareth-20, Petrolatum, Potassium Phosphate, Ceramide NP, Ceramide AP, Ceramide EOP, Carbomer, Dimethicone, Behentrimonium Methosulfate, Sodium Lauroyl Lactylate, Sodium Hyaluronate, Cholesterol, Phenoxyethanol, Disodium EDTA, Dipotassium Phosphate, Tocopherol, Phytosphingosine, Xanthan Gum, and Ethylhexylglycerin.
